(19) United States
(12) Patent Application Publication
(10) Pub. No.: US 2021/0113499 A1
(43) Pub. Date: Apr. 22, 2021

(54) CERAMIDE MIMICS FOR TREATMENT OF ALZHEIMER'S DISEASE

(71) Applicant: University of Kentucky Research Foundation, Lexington, KY (US)

(72) Inventor: Erhard Bieberich, Lexington, KY (US)

(21) Appl. No.: 17/074,344

(22) Filed: Oct. 19, 2020

Related U.S. Application Data
(60) Provisional application No. 62/923,195, filed on Oct. 18, 2019.

(51) Int. Cl. A61K 31/164; G01N 33/68

(52) U.S. Cl. CPC A61K 31/164; G01N 2800/52; G01N 2800/2821; G01N 33/6896

(57) ABSTRACT

The presently-disclosed subject matter generally relates to methods for diagnosing a subject with Alzheimer's Disease and treating the subject with an effective amount of a ceramide analogue. The presently disclosed matter further relates to methods for inhibiting protein binding to exosomes by administering ceramide analogues to a subject in need thereof. The presently disclosed matter also relates to methods of reducing the size of exosomes in a subject, comprising: administering an effective amount of a ceramide analogue to a subject in need thereof.

### RELATED APPLICATIONS

This application claims priority from U.S. Provisional Patent Application No. 62/923,195 filed on Oct. 18, 2019 the entire disclosure of which is incorporated herein by this reference.

### GOVERNMENT INTEREST

This invention was made with government support under grant number 2R01AG034389-06A1 awarded by the National Institutes of Health. The government has certain rights in the invention.

### TECHNICAL FIELD

The presently-disclosed subject matter generally relates to methods for diagnosing a subject with Alzheimer's Disease. The presently disclosed matter further relates to methods for inhibiting protein binding to exosomes by administering ceramide analogues to a subject in need thereof. Also disclosed are methods of reducing the size of exosomes in a subject comprising administering a ceramide analogue to a subject in need thereof.

### BACKGROUND

AP plaque deposits and tau neurofibrillary tangle formation are hallmarks of AD (Bertram, 2008; Serrano-Pozo, 2011). However, it is still controversial which of the two factors is critical for neuronal dysfunction and death, ultimately leading to cognitive decline and demise of the patient. Most of the previous studies assumed that the buildup of Aβ or tau by themselves induces neurotoxicity (Nisbet & Gotz, 2018; Rapoport, Dawson, Binder, Vitek, & Ferreira, 2002; Tu, Okamoto, Lipton, & Xu, 2014). This assumption, however, was in stark contrast to observations in AD mouse models and patients showing significant buildup of plaques and tangles without obvious neuronal cell death (Serrano-Pozo, Frosch, Masliah, & Hyman, 2011). It is believed that neurotoxicity of Aβ is mediated by its interaction with an unknown factor. Based on the previous studies showing that Aβ associates with astrocyte-derived exosomes (here termed astrosomes), this interaction was tested to determine if the interaction mediates neurotoxicity of Aβ (Dinkins, Dasgupta, Wang, Zhu, & Bieberich, 2014; Wang et al., 2012).

Exosomes are generated as intraluminal vesicles of multivesicular endosomes and secreted as a type of extracellular vesicles by a large variety of cells and tissues (Colombo et al., 2013; Colombo, Raposo, & Thery, 2014; Zhang, Liu, Liu, & Tang, 2019). Exosomes are deemed to serve as carriers for the intercellular transport of micro RNAs and some proteins. Although their size of 100 nm favors a high membrane surface-to-volume ratio, the role of membrane lipids in exosomes remains largely unexplored (Dinkins, Wang, & Bieberich, 2017; Elsherbini & Bieberich, 2018; Skotland, Sandvig, & Llorente, 2017). The sphingolipid ceramide is enriched in the membrane of astrosomes (Wang et al., 2012). Ceramide mediates association of Aβ with astrosomes and that this association leads to astrosome aggregation in vitro, a process suggested to nucleate amyloid plaques in AD brain (Dinkins et al., 2014). However, it is unknown if amyloid plaque nucleation is the only or even main function of astrosomes in vivo. Recent studies demonstrated that AP-associated exosomes cross the blood-brain-barrier and are detectable in serum from AD mice and patients (Fiandaca et al., 2015; Perez-Gonzalez, Gauthier, Kumar, & Levy, 2012; Sharples et al., 2008). In fact, exosomes purified from patient serum are proposed as AD biomarkers up to a decade prior to clinical symptoms of cognitive decline (Fiandaca et al., 2015). While a proportion of serum exosomes is clearly derived from brain, composition and function of these exosomes remains largely unknown. In the current study using mass spectrometry and anti-ceramide antibody, it was found that a proportion of brain-derived serum exosomes is enriched with the same ceramide species previously detected in astrosomes isolated from primary astrocyte culture (Dinkins et al., 2014). Glial fibrillary acidic protein (GFAP) and Aβ labeling confirmed their astrocytic origin and association with Aβ. These Aβ-associated astrosomes were taken up by neural cells and specifically transported to mitochondria, thereby inducing mitochondrial damage and caspase activation. Most importantly, the concentration of Aβ associated with astrosomes inducing damage was orders of magnitude lower than required when using Aβ without astrosomes. Aβ-associated astrosomes induced formation of a pro-apoptotic complex between Aβ and voltage-dependent anion channel 1 (VDAC1), the main ADP/ATP transporter in the outer mitochondrial membrane (Okada et al., 2004; Shoshan-Barmatz et al., 2010). These results suggest that astrosomes are the unknown factor mediating neurotoxicity of Aβ by inducing mitochondrial damage and apoptosis. The data also indicate that Aβ-associated exosomes may comprise a novel pharmacological target for AD therapy.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter generally relates to methods of diagnosing a subject with Alzheimer's Disease. The presently disclosed subject matter further relates to methods of inhibiting protein binding to exosomes comprising contacting exosomes with an effective amount of a ceramide analogue.

One embodiment of the present invention is a method of diagnosing and treating a subject with Alzheimer's Disease, said method comprising: a. obtaining a blood sample from a subject; b. purifying exosomes from the blood; c. detecting the size of exosomes in the blood; d. diagnosing the subject with Alzheimer's Disease when exosomes over 100 nm in size comprise at least 30% of the total population of exosomes; and e. administering an effective amount of a ceramide analogue to the subject diagnosed with Alzheimer's Disease. In a further embodiment of the present invention, the ceramide analogue is N-oleoyl serinol (S18). In some embodiments of the present invention, the effective amount is about 50 micromolar.

Another embodiment of the present invention is a method of diagnosing and treating a subject with Alzheimer's Disease, said method comprising: a. obtaining a blood sample from a subject; b. purifying exosomes from the blood; c. determining lipid composition of the exosomes in the subject; d. comparing the lipid composition of the exosomes in the subject to the lipid composition of the exosomes of healthy subjects; e. diagnosing the subject with Alzheimer's Disease when ceramide species C16:0 and C18:0 C22:0, C24:0 or C24:1 are elevated in the subject relative to the healthy subjects; and f. administering an effective amount of a ceramide analogue to the subject diagnosed with Alzheimer's Disease. In a further embodiment of the present invention, the ceramide analogue is N-oleoyl serinol (S18). In some embodiments of the present invention, the effective amount is about 50 micromolar.

Another embodiment of the present invention is a method of reducing the size of exosomes in a subject, comprising: administering an effective amount of a ceramide analogue to a subject in need thereof. In a further embodiment of the present invention, the ceramide analogue is N-oleoyl serinol (S18). In some embodiments of the present invention, the effective amount is about 50 micromolar. In a further embodiment, the size is reduced by inhibiting protein binding to the exosomes. In some embodiments, the protein is Aβ.

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description makes reference to the following drawings, wherein:

FIG. 1A shows 5×FAD serum-derived exosomes are enriched with ceramide and associated with GFAP. Cluster analysis of wild type (WT) and 5×FAD serum-derived exosomes after Nano Particle Tracking analysis showing different subpopulations within each preparation.

FIG. 1B shows 5×FAD serum-derived exosomes are enriched with ceramide and associated with GFAP. Cluster analysis of wild type (WT) and 5×FAD serum-derived exosomes after Nano Particle Tracking analysis showing different subpopulations within each preparation.

FIG. 1C shows 5×FAD serum-derived exosomes are enriched with ceramide and associated with GFAP. Immunoblot of exosome markers CD9, CD63, CD81, flotillin-1, and flotillin-2, demonstrating higher amounts of GFAP in 5×FAD exosomes compared to WT exosomes.

FIG. 1D shows 5×FAD serum-derived exosomes are enriched with ceramide and associated with GFAP. Ceramides species profile determined using LC-MS/MS of WT and 5×FAD serum-derived exosomes and normalized to phosphate content. Asterisks denote significance (p<0.05) after two-way ANOVA followed by Bonferroni correction (n=3).

FIG. 1E shows 5×FAD serum-derived exosomes are enriched with ceramide and associated with GFAP. Ceramides species profile determined using LC-MS/MS of WT and 5×FAD serum-derived exosomes and normalized to exosome count Asterisks denote significance (p<0.05) after two-way ANOVA followed by Bonferroni correction (n=3).

FIG. 2A shows 5×FAD serum astrosomes associated with Aβ form aggregates, which is reduced by the novel ceramide analog S18. Gel-electrophoresis after immune capturing of exosomes on beads using either ceramide antibody or control IgG and probing with anti-GFAP antibody.

FIG. 2B shows 5×FAD serum astrosomes associated with Aβ form aggregates, which is reduced by the novel ceramide analog S18. Dot-blot against Aβ using flow through for the same experiment.

FIG. 2C shows 5×FAD serum astrosomes associated with Aβ form aggregates, which is reduced by the novel ceramide analog S18. Size distribution of WT, 5×FAD, and 5×FAD exosomes treated with anti-ceramide IgG.

FIG. 2D shows 5×FAD serum astrosomes associated with Aβ form aggregates, which is reduced by the novel ceramide analog S18. Size distribution of WT, 5×FAD, and 5×FAD exosomes treated with the novel ceramide analog S18. Particle diameter of each sample is represented as ±SEM, two-way ANOVA, *p≤0.05.

FIG. 3A shows 5×FAD serum-exosomes contain astrosomes that are taken up by neural cells. Representative images of N2a cells incubated with exosomes isolated from WT and coimmunolabeled with antibodies against GFAP and ceramide.

FIG. 3B shows 5×FAD serum-exosomes contain astrosomes that are taken up by neural cells. Representative images of N2a cells incubated with exosomes isolated from 5×FAD serum and coimmunolabeled with antibodies against GFAP and ceramide.

FIG. 3C shows 5×FAD serum-exosomes contain astrosomes that are taken up by neural cells. Representative images of N2a cells incubated with exosomes isolated from WT. The Pearson's correlation coefficient was calculated to compare colocalization of GFAP and ceramide in WT (open bar) and 5×FAD (closed bar). Welch's t-test, *p≤0.05.

FIG. 3D shows 5×FAD serum-exosomes contain astrosomes that are taken up by neural cells. Representative images of N2a cells incubated with exosomes isolated from WT. Representative images of N2a cells incubated with exosomes isolated from WT and coimmunolabeled with antibodies against flotillin-2 and Aβ.

FIG. 3E shows 5×FAD serum-exosomes contain astrosomes that are taken up by neural cells. Representative images of N2a cells incubated with exosomes isolated from 5×FAD serum and coimmunolabeled with antibodies against flotillin-2 and Aβ.

FIG. 3F shows 5×FAD serum-exosomes contain astrosomes that are taken up by neural cells. Representative images of N2a cells incubated with exosomes isolated from WT. The Pearson's correlation coefficient was calculated to compare colocalization of flotillin-2 and Aβ signals in WT (open bar) and 5×FAD (closed bar). Welch's t-test, *p≤0.05.

FIG. 4A shows Serum-derived exosomes from 5×FAD mice and AD patients shuttle Aβ to mitochondria. Immunofluorescence images of N2a cells incubated with healthy control patient serum-derived exosomes labeled with anti-ceramide and flotillin-2 antibodies.

FIG. 4B shows Serum-derived exosomes from 5×FAD mice and AD patients shuttle Aβ to mitochondria. Immunofluorescence images of N2a cells incubated with AD patient serum-derived exosomes labeled with anti-ceramide and flotillin-2 antibodies.

FIG. 4C shows N2a cells were incubated with WT exosomes adn immunolabeled for flotillin-2 and mitochondrial marker Tom-20.

FIG. 4D shows N2a cells were incubated with 5×FAD exosomes and immunolabeled for flotillin-2 and mitochondrial marker Tom-20.

FIG. 4E shows Human neural progenitor cells incubated with control healthy human exosomes showing that only AD exosomes shuttle Aβ to neuron mitochondria.

FIG. 4F shows Human neural progenitor cells incubated with AD exosomes showing that only AD exosomes shuttle Aβ to neuron mitochondria.

FIG. 4G shows Ceramide fluorescence intensity in N2a cells incubated with WT or 5×FAD exosomes.

FIG. 4H shows Pearson's correlation calculation for colocalization of flotillin-2 and Tom-20 signals Aβ, Welch's t-test, *p≤0.05, **p≤0.01.

FIG. 4I shows Pearson's correlation calculation for colocalization of flotillin-2 and Tom-20 signals Tom-20, Welch's t-test, *p≤0.05, **p≤0.01.

FIG. 5A shows Neurotoxic effect of Aβ42/astrosome complexes on primary neuronal cultures. Representative single-focal-plane images of β-tubulin labeling obtained with control, A042, astrosome, or Aβ42/astrosome-incubated primary cultured mouse neuron.

FIG. 5B shows Neurotoxic effect of Aβ42/astrosome complexes on primary neuronal cultures. Average normalized density of β-tubulin labeling reveals that the greatest loss occurs in cultures treated with Aβ42/astrosome complexes. Asterisks at the bars corresponding to different treatment conditions indicate significant difference from control (one-way ANOVA with SNK post hoc test). ***p<0.001, “p<0.005, *p<0.05).

FIG. 5C shows Neurotoxic effect of Aβ42/astrosome complexes on primary neuronal cultures. TUNEL assay detected a 2.6-fold increase in neuronal cell death when Aβ42 and astrosomes were combined (one-way ANOVA with SNK post hoc test).

FIG. 6A shows 5×FAD exosomes induce mitochondrial clustering and mediate complex formation between Aβ and mitochondrial VDAC1. (A) Representative immunofluorescence images of N2a cells incubated with wild type exosomes (top panel) or 5×FAD exosomes (lower panel) showing increased number of PLA signals in cells incubated with 5×FAD exosomes. Each red dot denotes complex formation between Aβ and mitochondrial VDAC1.

FIG. 6B shows 5×FAD exosomes induce mitochondrial clustering and mediate complex formation between Aβ and mitochondrial VDAC1. (B) Calculation and comparison of average PLA signals per cell between wild type and 5×FAD incubations.

FIG. 6C shows 5×FAD exosomes induce mitochondrial clustering and mediate complex formation between Aβ and mitochondrial VDAC1. (C) Colocalization between PLA signal and mitochondrial marker Tom-20 staining, confirming that the complex formation occurs at mitochondria.

FIG. 6D shows 5×FAD exosomes induce mitochondrial clustering and mediate complex formation between Aβ and mitochondrial VDAC1. (D) N2a cells incubated with Vybrant CM DiI labeled exosomes from 5×FAD (top panel) or wild type (lower panel) serum showing mitochondrial clustering in 5×FAD exosome treated cells.

FIG. 6E shows 5×FAD exosomes induce mitochondrial clustering and mediate complex formation between Aβ and mitochondrial VDAC1. (E) Western blot of isolated mitochondrial proteins against Drp-1 antibody using VDAC1 as a reference protein.

FIG. 7A shows 5×FAD and human AD patient serum-derived exosomes trigger apoptosis in cells induced by interaction between mitochondrial VDAC1 and Aβ. Representative immunofluorescence images of N2a cells incubated with 5×FAD exosomes. Flica assays were followed by PLAs. Images show that cells with higher signal number of PLA undergo apoptosis.

FIG. 7B shows 5×FAD and human AD patient serum-derived exosomes trigger apoptosis in cells induced by interaction between mitochondrial VDAC1 and Aβ. Human AD patient serum-derived exosomes. Flica assays were followed by PLAs. Images show that cells with higher signal number of PLA undergo apoptosis.

FIG. 7C shows 5×FAD and human AD patient serum-derived exosomes trigger apoptosis in cells induced by interaction between mitochondrial VDAC1 and Aβ. (C) Western blot with N2a cell lysate immunostained for cleaved-caspase 3 using GAPDH as a reference protein.

FIG. 7D shows 5×FAD and human AD patient serum-derived exosomes trigger apoptosis in cells induced by interaction between mitochondrial VDAC1 and Aβ. (D) Relative fold expression of cleaved-caspase-3 normalized to GAPDH. One-way ANOVA followed by Tukey correction, **P≤0.001.

FIG. 8 shows Potential mechanism of neurotoxicity induced by Aβ-associated astrosomes. Aβ secreted by neurons (red) binds to ceramide-enriched exosomes secreted by astrocytes (astrosomes, green). Aβ-associated astrosomes are endocytosed by neurons and transported to mitochondria. The vesicles fuse with the outer mitochondrial membrane and mediate binding of Aβ to VDAC1. A pro-apoptotic pore is formed that leads to activation of caspases and induction of neuronal cell death.

FIG. 9A shows Serum-derived exosomes from AD patients are enriched with ceramide. (A) Ceramide species profile using lipid mass spectrometry (LC-MS/MS) of AD patient serum-derived exosomes normalized to phosphate content.

FIG. 9B shows Serum-derived exosomes from AD patients are enriched with ceramide. (B) Immunoblot for exosome markers CD63 and Flotillin-1 showing equal protein expression levels of GFAP in AD and healthy control individuals.

FIG. 9C shows Serum-derived exosomes from AD patients are enriched with ceramide. (C) Structures of ceramide analogs S18 and B16.

FIG. 10A shows Serum derived exosomes from WT and 5×FAD mice are taken up by N2a cells. Representative fluorescence microscopy images of PKH67-labeled exosomes from WT mice showing their uptake by N2a cells.

FIG. 10B shows Serum derived exosomes from WT and 5×FAD mice are taken up by N2a cells. Representative fluorescence microscopy images of PKH67-labeled exosomes from 5×FAD(B) mice showing their uptake by N2a cells.

FIG. 11A shows 5FAD exosomes retained complex formation between Aβ and ceramide after uptake into N2a cells. WT exosomes were labeled with PKH67 dye and then then used for incubation of N2a cells. PLA shows complex formation between Aβ and ceramide only with 5×FAD exosomes.

FIG. 11B shows 5×FAD exosomes retained complex formation between Aβ and ceramide after uptake into N2a cells. 5×FAD serum-derived exosomes were labeled with PKH67 dye and then then used for incubation of N2a cells. PLA shows complex formation between Aβ and ceramide only with 5×FAD exosomes.

FIG. 12A shows Interaction between Aβ and mitochondrial via VDAC1 in human brain. (A) Representative fluorescence image of human brain section showing colocalization of Aβ with mitochondrial Tom-20 around amyloid plaque (arrows).

FIG. 12B shows Interaction between Aβ and mitochondrial via VDAC1 in human brain. PLA using antibodies against Aβ and mitochondrial VDAC1 showing complex formation in cells surrounding amyloid plaque.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

Following long-standing patent law convention, the terms “a”, “an”, and “the” refer to “one or more” when used in this application, including the claims. Thus, for example, reference to “a biomarker” includes a plurality of such biomarkers, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, width, length, height, concentration or percentage is meant to encompass variations of in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a disorder such as Alzheimer's Disease” means having been subjected to a physical examination by a person of skill, for example, a physician, and found to have a condition that can be diagnosed or described as Alzheimer's Disease.

As used herein, the term “subject” refers to a target of administration. The subject of the herein disclosed methods can be a mammal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A “patient” refers to a subject afflicted with a disease or disorder. The term “patient” includes human and veterinary subjects.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level if or any particular patient will depend upon a variety of factors including the disorder being treated and the severity of the disorder; the specific composition employed; the age, bodyweight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

As used herein, ceramide analogue refers to beta-hydroxylamine, hydroxylated, or amide ceramide analogue as commonly known in the art. For example, FTY720 (fingolimod), phytoceramide, S16 or B16.

### EXAMPLES

Materials and Methods

Cell Cultures

The N2a cell line was obtained from ATCC (CCL-131™). The cells were grown to 90% confluence at 37.0 and 5% CO2 atmosphere in Dulbecco's modified Eagle's medium (DMEM) (Gibco, Invitrogen, CA, USA) supplemented with 10% fetal bovine serum (FBS) on 100 mm plates (Corning, MA, USA). For immunocytochemistry analyses, cells were seeded on poly-L-lysine (Milipore-Sigma, Montana, USA) coated cover slips at 10,000 cells/cover slip. Cells were gradually deprived of serum to allow for differentiation into neuron-like cells. To cultivate human induced pluripotent stem (iPS) cell-derived neuroprogenitor (NP) cells, the ReNcell VM Human NP cell line was obtained from Millipore ((Temecula, Calif., USA, Cat #SCC008). Cells were maintained according to the supplier's protocol. Briefly, cells were expanded on laminin-coated 100 mm tissue culture dishes (Corning) in ReNcell NSC maintenance medium (Millipore) supplemented with 20 ng/mL fibroblast growth factor-2 (FGF-2) and 20 ng/mL epidermal growth factor (EGF) (Millipore). The medium was changed daily during the maintenance period. The cells were passaged once a week using Accutase (Millipore). Cells were then differentiated by seeding them at around 60% confluency on freshly laminin-coated dishes and growing overnight in the presence of growth factors, followed by withdrawal of growth factors. The media were replaced every other day up to 10 days during the differentiation period.

Serum Exosome Isolation, Quantification, and Labeling

Sera were isolated from freshly obtained mouse blood. Blood was drawn through heart puncture and was allowed to clot at room temperature for 30 min. Blood was then centrifuged at 1,800×g for 10 min at 4° C. The clear upper layer was transferred to a fresh tube and centrifuged at 3,000×g for 15 min to pellet residual blood cells. Exosomes were extracted using ExoQuick exosome solution (EXOQ; System Biosciences, Inc., Mountain View, Calif., USA) according to the manufacturer protocol. Briefly, one-fourth milliliter aliquots of serum was treated with 67 μl of ExoQuick exosome solution, followed by incubation for 60 min at 4° C. to precipitate total exosomes. Tubes were then centrifuged at 1,500×g for 30 min. Each exosome pellet was resuspended in 100 μl of PBS with 1× Halt™ Protease Inhibitor Cocktail (Thermo Fisher, Massachusetts, USA). In certain experiments exosomes were labeled with PKH67 Green Fluorescent Dye. The exosomes were labeled with PKH67 Green Fluorescent Cell Linker Kit for General Cell Membrane Labelling (Sigma-Aldrich) according to the manufacturer's protocol. Briefly, ExoQuick pellets were resuspended in PBS, 1 ml of Diluent C (CGLDIL, Sigma-Aldrich) was then added to each sample. As a control, 1 ml of Diluent C after adding the same volume of PBS was used. Next, 4 μl of PKH67 dye was added to 1 ml of Diluent C then mixed with the exosomes and the control, PKH67/Diluent C mixture was ultra-centrifuged before being added to samples. The samples were allowed to incubate <5 min on a rotor plate. One ml of 1% BSA was then added to bind excess dye. Samples were ultra-centrifuged at 110,000×g for 70 min, washed and centrifuged again. For exosomes quantification, nanoparticle tracking analysis (NTA) with the ZetaView PMX110 (Particle Metrix) was used. Briefly, exosomes were resuspended in PBS. Two ml of appropriately diluted samples were injected into the ZetaView cell. The instrument was set to obtain NTA measurements at 11 positions, two cycles at each position. During acquisition, temperature was set to 23° C., camera sensitivity to 82, 30 frames/s, and shutter speed to 250. Polystyrene beads (100 nm) were used for instrument calibration. For exosome incubation with ceramide analogs N-oleoyl serinol (S18 or bis palmitoyl ethanolamine (B16) the exosomes prepared from 5×FAD or control serum were incubated at 37° C. for 16 h with 50 μM S18 or B16.

Besides ExoQuick exosome isolation method, Exoeasy Maxi kit was used (Qiagen, Germany) to isolate exosomes from sera following the manufacturer protocol. Briefly, sera were diluted with an equal volume of distilled water to reduce viscosity and they were passed through a 0.45 μm filter to remove larger particles. 1 volume of Exoeasy binding buffer (XBP) was then added to 1 volume of sample. Sample/XBP mix was then added onto the Exoeasy spin column and centrifuge at 500×g for 1 min. Flow-through was discarded and the columns were placed back into the same collection tube. 10 ml buffer Exoeasy washing buffer (WP) were then added to columns, followed by centrifugation at 5000×g for 5 min to remove residual buffer from the column. Flow-through together with the collection tube were then discarded. Spin columns were then transferred to fresh collection tubes. 400 μl of elution buffer were added to the membrane and incubated for 1 min, followed by centrifugation at 500×g for 5 min to collect the eluate.

Immunocytochemistry

N2a or human NP cells were seeded on poly-L-lysine coated cover slips at a density of 25,000 cells/cover slip. N2a cells were allowed to differentiate by gradual serum deprivation (Fenteany, Standaert, Reichard, Corey, & Schreiber, 1994). Two days prior to exosome incubation, exosome-free FBS (EXO-FBS—System Biosciences, Mountain View, Calif., USA) was used to supplement the media. Cells were then incubated with exosomes and washed three times with PBS, followed by fixation with 4% p-formaldehyde containing 0.5% glutaraldehyde in PBS for 15 min at room temperature. Permeabilization was performed by incubation with 0.2% Triton X-100 in PBS for 5 min at room temperature. Non-specific binding sites were blocked with 3% ovalbumin/PBS for 1 h at 37° C. Cells were then incubated with primary antibodies at 4° C. overnight. The next day, cells were washed with PBS and incubated with secondary antibodies diluted 1:300 in 0.1% ovalbumin/PBS for 2 h at 37° C. Secondary antibodies were Cy2-conjugated donkey anti-mouse IgM, Alexa Fluor 546-conjugated donkey anti-rabbit IgG, and Alexa Fluor 647-conjugated goat anti-mouse IgG (Jackson ImmunoResearch, West Grove, Pa.). After washing, cover slips were mounted using Fluoroshield supplemented with DAPI (Sigma-Aldrich) to visualize the nuclei. The following primary antibodies were used: anti-ceramide rabbit IgG (1:100, present inventors), anti-flotillin-2 mouse IgG (1:300 BD Biosciences, California, USA, 610383), anti-amyloid-beta mouse IgG 4G8 clone (1:200 Biolegends, California, USA, SIG-39220), anti-GFAP mouse IgG (1:500, abcam, Cambridge, Mass., USA, ab10062), anti-Tom 20 rabbit IgG (1:200, Santa Cruz, sc-11415), anti-VDAC1 rabbit IgG (1:500, Abcam, ab15895). Fluorescence microscopy was performed using Eclipse Ti2-E inverted microscope system (Nikon, New York, USA). Images were processed using Nikon NIS-Elements software equipped with a 3D deconvolution program. Pearson's correlation coefficient for two fluorescence channels in overlays was used to assess the degree of colocalization.

Proximity Ligation Assay

Cells were grown and treated as described above in the protocol for immunocytochemistry. Non-specific binding sites were blocked with Duolink PLA blocking solution (Sigma-Aldrich) for 1 h at 37° C. The primary antibodies used were; anti-Aß mouse IgG (1:500 4G8, Biolegends, California, USA, SIG-39220), anti-VDAC1 rabbit IgG (1:1000 abcam, Cambridge, Mass., USA, ab34726) Secondary PLA probes: anti-mouse MINUS affinity-purified donkey anti-mouse IgG (H+L) and anti-rabbit PLUS affinity-purified donkey anti-rabbit IgG (H+L) were diluted 1:5 in antibody diluent buffer and samples incubated for 1 h at 37° C. followed by ligation and amplification steps as described in the manufacturer's protocol (Duolink, Sigma-Aldrich). Cover slips were mounted using Fluoroshield supplemented with DAPI (Sigma-Aldrich) to visualize the nuclei. Images obtained with secondary antibody only were used as negative controls representing the background intensity in a laser channel. ImageJ software (https://imagej.nih.gov/ij/) was used to analyze the pictures. Two channels (DAPI and TRITC) were separated to analyze nuclear staining (DAPI) of the images separately from the TRITC-channel associated with the PLA dots.

Firstly, a threshold was set in order to identify nucleus and to allow for binary conversion (black and white). Morphological function was used to separate touching nuclei. Nuclei were counted and added to the region of interest (ROI) where the appropriate minimum and maximum pixel area sizes were set. On the other channel, the number of dots (PLA signals) in each cell as identified by labeling of nuclei was calculated with the “Measure” command from the ROI manager using single point as an output type.

Isolation of Mitochondria

N2a cells were seeded on 100 mm dishes at 35-40% of density, followed by incubation with wild type or 5×FAD serum exosomes. Sixteen hours later, cells were harvested and washed twice with ice-cold PBS. Cell pellets were then transferred into a Dounce homogenizer and disrupted with 2 ml of ice-cold mitochondria extraction buffer [10 mM HEPES, 125 mM sucrose, 0.01% BSA, 250 mM mannitol, 10 mM EGTA, and protease inhibitors (pH 7.2)]. The homogenates were transferred into a centrifuge tube and cell debris pelleted at 700×g at 4° C. for 10 min to enrich for mitochondria. Following centrifugation under same conditions, supernatants were transferred to a new ice-cold tube, and then mitochondria pelleted at 10,000×g for 15 min at 4° C. The mitochondrial pellet was resuspended in 1 ml of lipid binding buffer [20 mM Tris-HCl, 150 mM NaCl, 1 mM EDTA (pH 7.5), and 1% digitonin, supplemented with protein inhibitor cocktail (Roche)]. Complete lysis of mitochondrial membranes was achieved by sonication. Removal of insoluble debris was achieved by centrifugation at 10,000×g for 15 min at 4° C. The protein concentration in the supernatants from untreated cells and treated cells was determined using Bio-rad RC DC™ Protein Assay.

FLICA Assay

The FLICA 660 Poly Caspase Assay Kit (ImmunoChemistry Technologies, Minnesota, USA) was used to determine the presence of early caspase activation. This in vitro assay employs the fluorescent inhibitor probe 660-VAD-FMK to label active caspase enzymes in living cells. N2a cells were incubated with exosomes (5,000-10,000 exosomes/cell) for 6 h at 37° C. The cells were washed twice with PBS and resuspended in RPMI medium with 10% FBS before staining with 30×FAM-VAD-FMK for 30 min at 37° C. Cells were washed with 1× apoptosis wash buffer prior to being fixed with 4% paraformaldehyde supplemented with 0.5% glutaraldehyde. The assay was then followed by PLA as described above.

Western Blot and Dot Blot

For Western blot analysis, samples were mixed with an equal volume of 2×Laemmli sample

buffer. Samples were resolved by SDS gel electrophoresis on polyacrylamide gels and transferred to nitrocellulose membrane (Hybond ECL, Amersham Biosciences, UK). Non-specific binding sites were blocked with 5% fat-free dry milk in PBS containing 0.05% Tween-20 followed by overnight incubation with primary antibodies. For exosome characterization CD9, CD63, CD81 rabbit antibodies from ExoAb Antibody Kit (System Biosciences, Inc., Mountain View, Calif., USA) after dilution to 1:1000 were used. The following primary antibodies were used for immmunolabeling on Western blots: anti-flotillin-2 mouse IgG (1:1000, BD Biosciences, California, USA, 610383), anti-cleaved caspase-3 rabbit IgG (Cell Signaling, Danvers, Mass., USA, #9664), anti-VDAC1 goat polyclonal IgG (1: 200, Santa Cruz Biotechnology, Inc., CA, USA), anti-Drp-1 mouse IgG1 kappa light chain (Santa Cruz, Dallas, Tex., USA, sc-271583). Signals were detected using either pico or femto chemiluminescent (ECL) horseradish peroxidase (HRP) substrate (Thermo Fisher, Massachusetts, USA). Blot images were developed using Azure c600 system (Azure Biosystems, California, USA).

Exosome Immune Capturing on Beads: Affinity Purification Using Ceramide Beads

Twenty μL of protein A sepharose conjugated magnetic beads were pre-blocked with FcR Blocking Reagent (MACs Miltenyi Biotec) for 1 h at room temperature. After 3-times washing with lipid biding buffer [20 mM Tris-HCl, 150 mM NaCl, 1 mM EDTA (pH 7.5)], either anti-ceramide rabbit IgG or control non-specific rabbit IgG were immobilized on the beads in 1% BSA. Approximately 2 μg were added to each sample and the reaction kept mixing overnight on a rotary plate. Next day, beads were washed 3-times and diluted exosome samples were added and allowed to incubate with the beads for 2 h at room temperature. Beads were then collected using magnetic columns and washed 3-times with detergent free lipid binding buffer. The beads were incubated with an adequate volume of 2×sample Laemmli buffer, heated at 90° C. for 10 min and processed for immunoblot labeling of GFAP. Aliquots of the flow through were used for dot blots determining Aß content and the residual sample processed for Western blot using 4× sample Laemmli buffer. Equal volumes of the samples were then applied to each well for Western blot analysis. 4 μL were used for dot blot with the flow through of each sample.

Mass Spectrometric Analysis of Lipids

Exosomes prepared from serum were taken up in water and ceramide species were quantified in the sphingolipidomics (LC-MS/MS) analysis core facility at the Medical University of South Carolina, Charleston, S.C. The lipid concentration was normalized to lipid phosphate and exosome number.

Statistical Analysis

Clustering analyses were performed with Particle Explorer V2.1.4 (Particle Metrix Inc., Germany) using the following features (1. Particle size 2. Position 3. Area std 4. Mean intensity std 5. Trajectory total distance, std speed, track time, med-speed, and max-speed). For the lipid analysis, results were analyzed with Two-way ANOVA using ceramide species and genetic background as two independent factors. All other data were analyzed by unpaired t-test with Welch's correction. Results showing p<0.05 were reported as statistically significant. All statistical analysis were done on Graphpad prism software.

### Results

### Example 1: 5×FAD Mouse and AD Patient Serum Contains Exosomes Enriched with Ceramide and Derived from Astrocytes (Astrosomes)

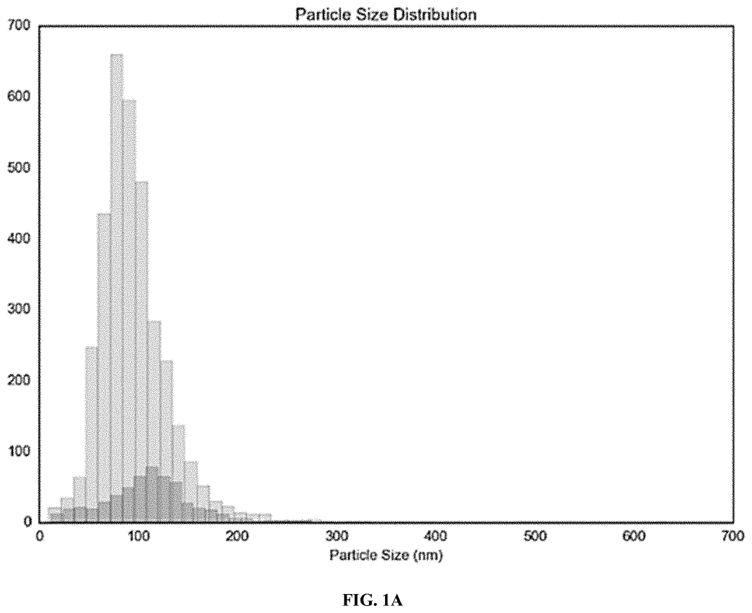

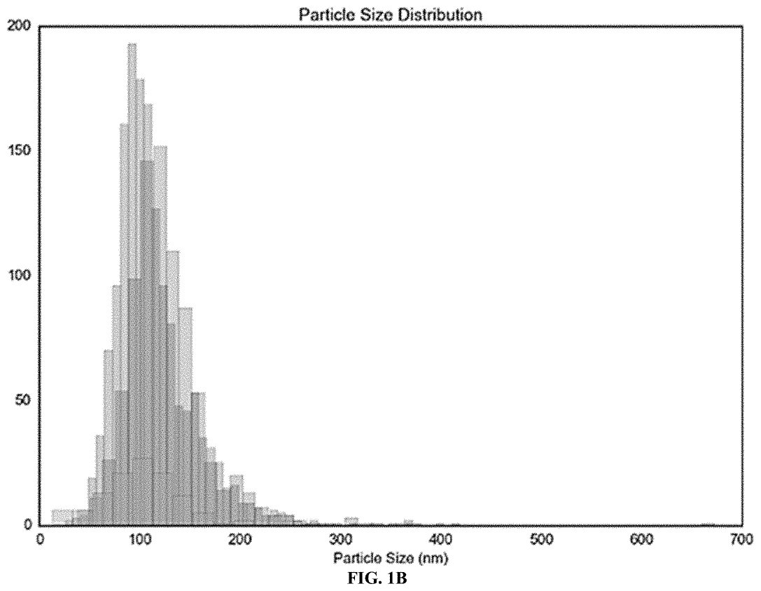

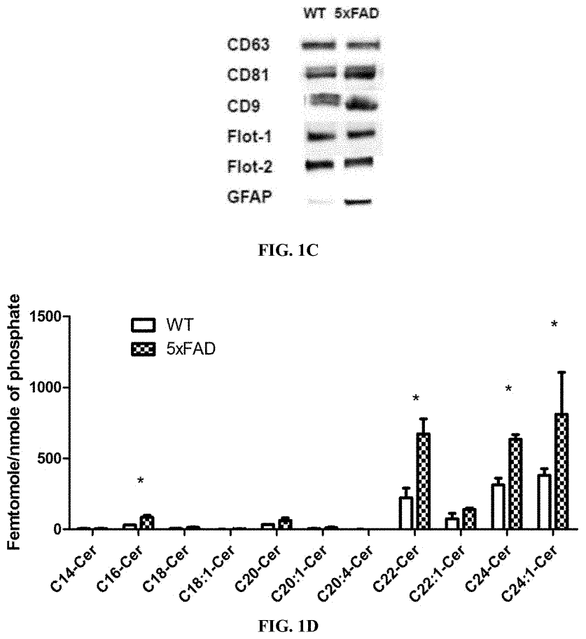

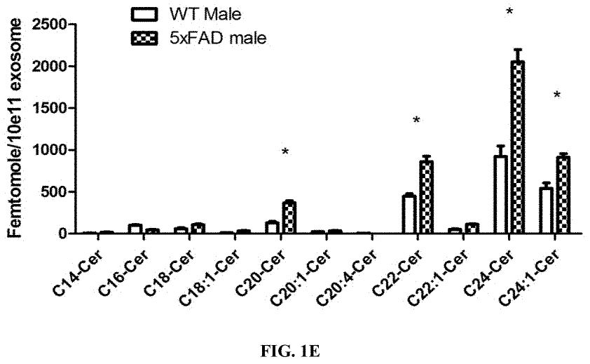

Several studies showed that exosomes cross the blood-brain-barrier (BBB) carrying toxic and misfolded protein of CNS origin (Fiandaca et al., 2015; Shi et al., 2014). These studies also showed that purification of exosomes from blood, serum, or blood allows characterization of exosomes from different cell types in the brain, including astrocytes. Polymer precipitation and membrane affinity chromatography were used to isolate exosomes from sera of transgenic mouse model of AD and AD patients as it was shown to give consistent results (Enderle et al., 2015; Helwa et al., 2017; Taverna et al., 2015). Due to the limitations in availability of AD patient serum the experiments were first focused on characterization of exosomes prepared from serum of the transgenic mouse model of AD (5×FAD) and wild type littermates with identical genetic background (C57Bl/6). 5×FAD mice overexpress presenilins (PS1) with two FAD mutations (M146L and L286V) as well as amyloid precursor protein (APP) with three FAD mutations (V717I, I716V, and K670N/M671L,){Oakley, 2006 #680}. Nanoparticle tracker analyses (NTA, Zetaview) and cluster analyses software (Particle Explorer, Particle Metrix, Mebane, N.C.) showed that the majority of exosomes from wild type serum was composed of a homogenous population of vesicles with medium size of 100 nm (FIG. 1A), while exosomes from 5×FAD serum contained an additional vesicle population of larger sizes accounting for 37±4% of the total population, indicating aggregate formation (FIG. 1B). Immunoblot analysis was used to validate the presence of exosomal markers such as tetraspanin proteins (CD63, C9, and CD81) as well as raft and exosome-associated proteins flottilin-1 and flottilin-2, and the astrocyte marker GFAP (FIG. 1C).

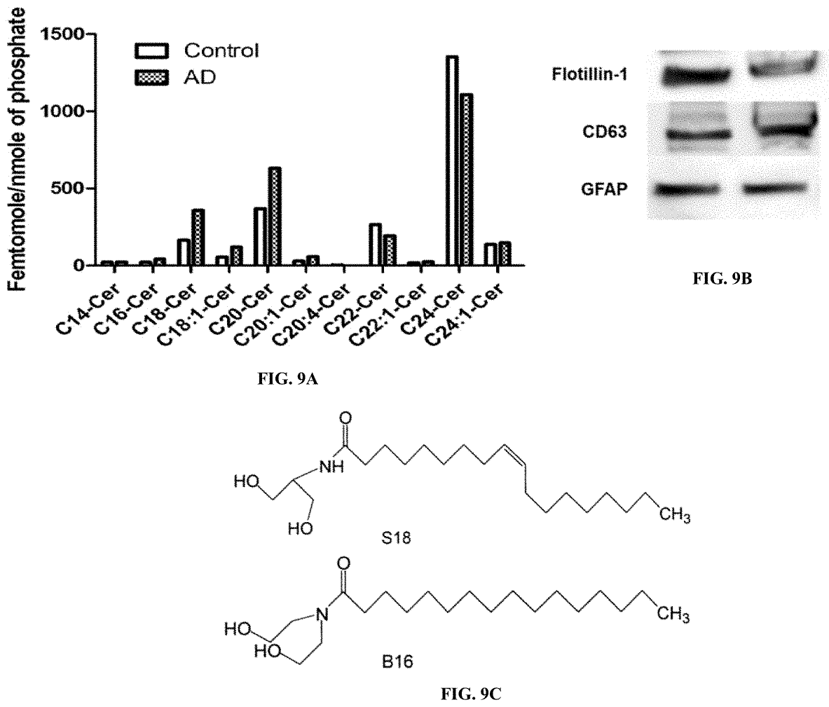

Lipid analysis using mass spectrometry (LC-MS/MS) showed that 5×FAD exosomes were enriched with ceramide, particularly C16:0, C18:0, C22:0, C24:0, and C24:1 ceramide (FIGS. 1D and E). Normalization to lipid phosphate (FIG. 1D) as well as particle count (FIG. 1E) showed similar enrichment, confirming that the ceramide composition was representative for the exosome population in serum. Ceramide composition and GFAP association of exosomes in serum from AD patients was also determined. FIG. 9A shows that the levels of some of ceramide species (C16:0 and C18:0 ceramide) were increased in AD patient exosomes, while others (C22:0 and C24:0 ceramide) were not. The GFAP level associated with serum exosomes from AD patients was comparable to that of healthy controls (FIG. 9B), suggesting that the main difference to serum exosomes from controls is the enrichment with ceramide. Example 2: Serum astrosomes are associated with Aβ and sensitive to novel ceramide analogs

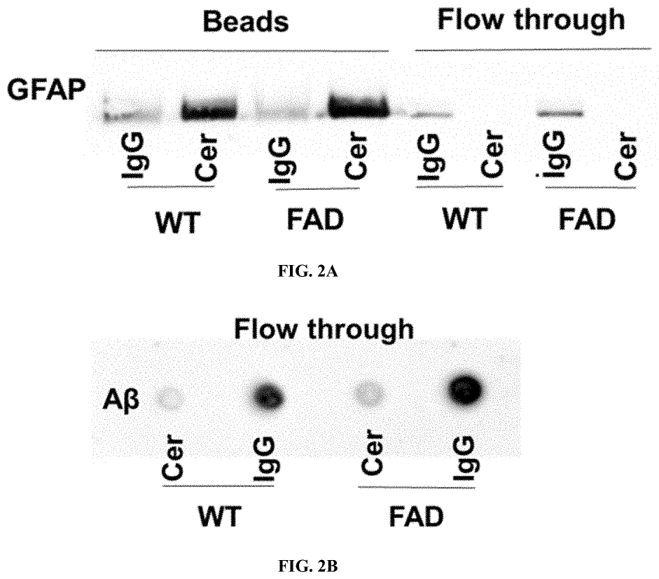

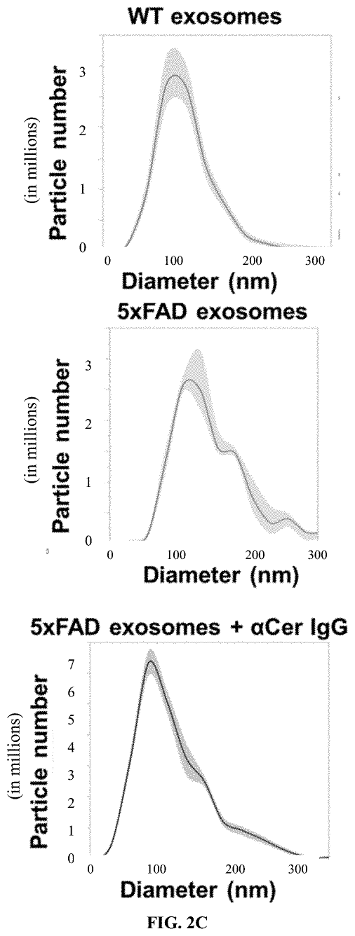

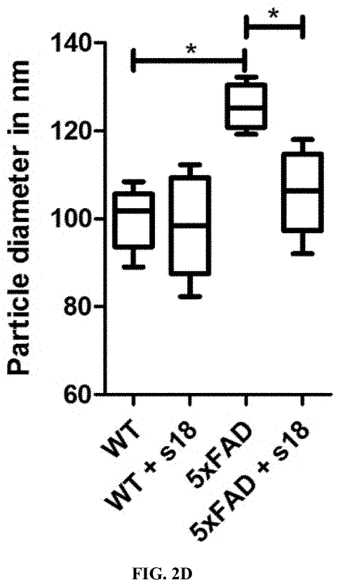

To further characterize ceramide-enriched exosomes, anti-ceramide rabbit IgG immobilized on protein A sepharose beads were used to separate ceramide-enriched exosomes from other exosome populations in serum. FIG. 2A shows that GFAP labeling was only found with exosomes bound to the beads, while exosomes in the flow through were GFAP negative. Control rabbit IgG did not bind any serum-derived exosomes confirming specificity of the binding reaction for ceramide-enriched astrosomes. Wild type serum also contained astrosomes retained by anti-ceramide antibody, however, at lower concentration as indicated by weaker immunolabeling for GFAP. NTA analysis showed that retention by anti-ceramide beads reduced the number of exosomes by 2.33% from wild type and 9.2% from 5×FAD serum indicating that the concentration of astrosomes is 5×FAD serum is ≃4-fold higher than that in wild type serum. Next, it was determined if ceramide-enriched exosomes in serum were associated with Aß by determining the amount of Aß retained on anti-ceramide beads vs. that in flow through. Immunolabeling using dot blots showed that only the flow through of beads with control IgG contained Aß42, while amyloid peptide was retained on anti-ceramide beads (FIG. 2B). Consistent with immunolabeling for GFAP, the amount of Aß was 2.2-fold higher in 5×FAD serum than that from wild type mice. Since ceramide-enriched exosomes were associated with GFAP as well as Aß, 5×FAD serum contained a portion of astrosomes enriched with ceramide and associated with Aß.

Enrichment of astrosomes with ceramide suggested that this lipid participates in association of Aß to astrosomes. This hypothesis is consistent with previous studies showing that anti-ceramide IgG prevented aggregation of exosomes induced by incubation with Aβ (Dinkins et al., 2014). FIG. 2C shows that incubation with anti-ceramide IgG abolished the proportion of larger sized vesicles in the preparation of 5×FAD exosomes, similar to the effect of anti-ceramide antibody on aggregation of Aβ-associated astrosomes derived from cell culture media. Vesicle size was reduced by 17% when adding the novel ceramide analogs N-oleoyl serinol (S18) but not N-palmitoyl bisethanolamine (B16, structures are shown in supplement FIG. 1 C) to 5×FAD exosomes, suggesting that S18 is a ceramide mimic that disrupts Aβ association and aggregation of astrosomes, probably by interfering with the ceramide-mediated binding of Aβ to astrosomes.

### Example 3: Astrosomes Transport Aß and Ceramide to Mitochondria

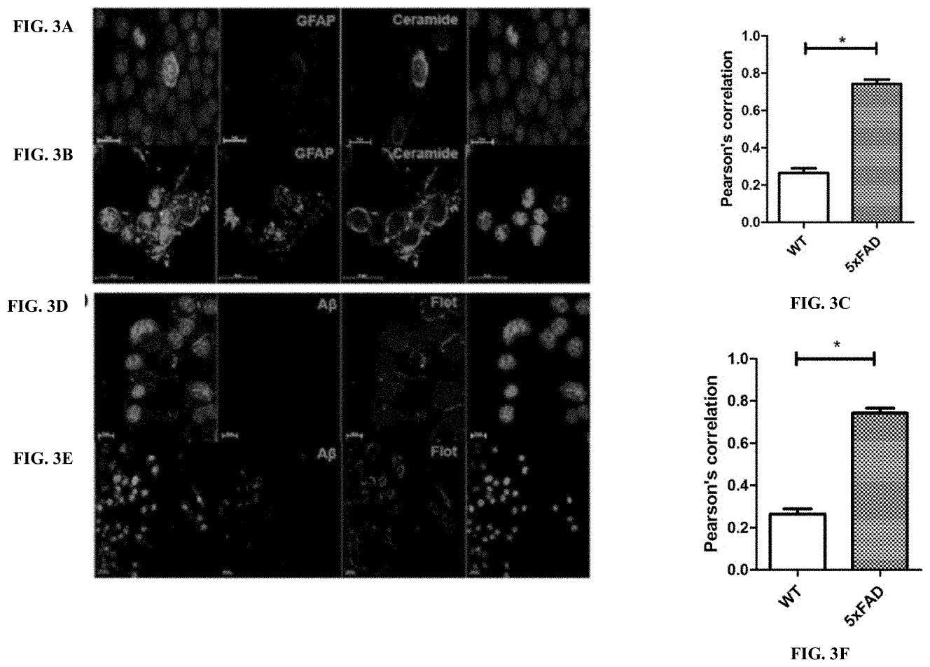

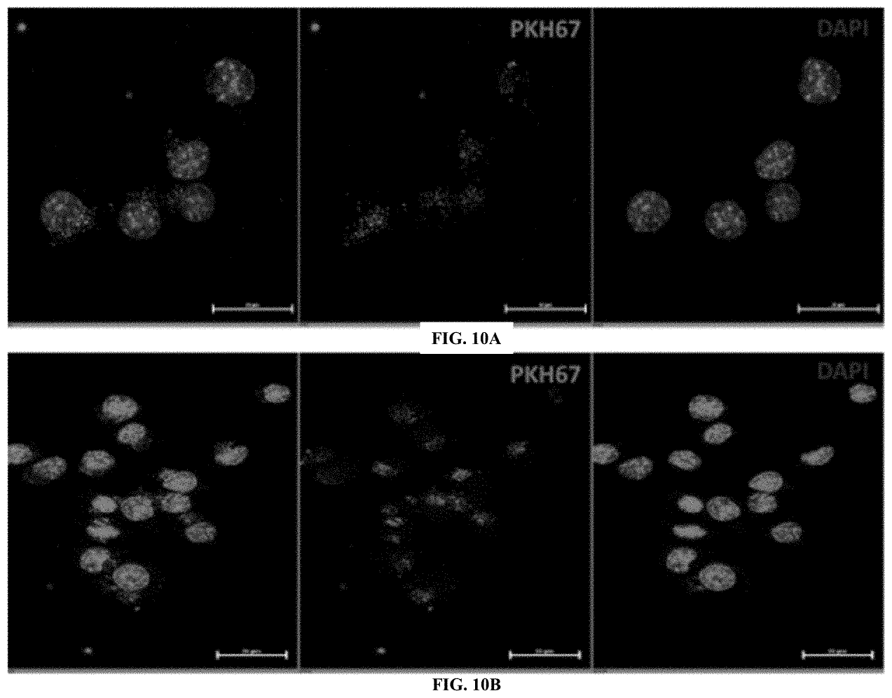

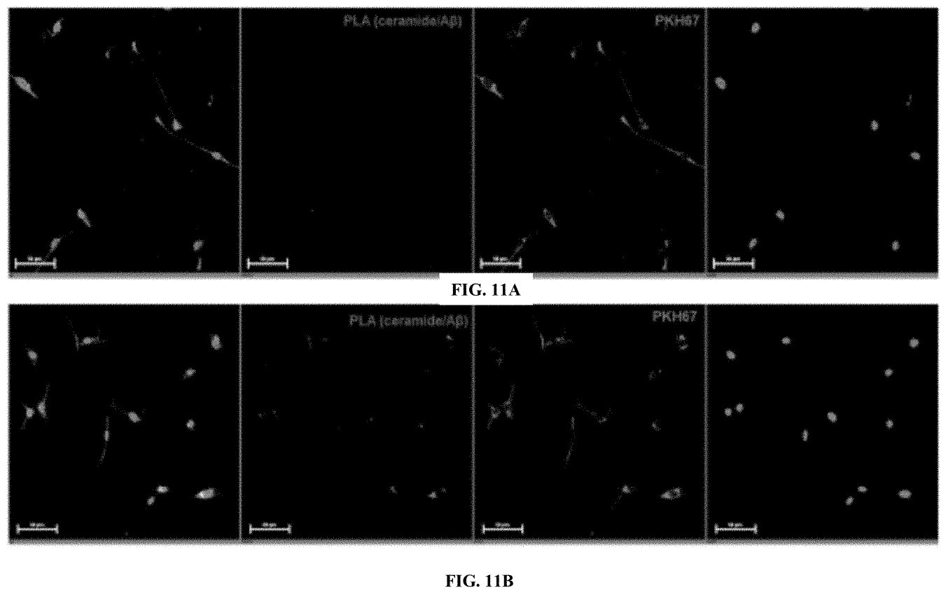

To test if serum-derived exosomes are up taken by neural cells, neuronally differentiated N2a cells were incubated with exosomes labeled with the fluorescent membrane-binding dye PKH67. FIGS. 10A and B shows that both, WT and 5×FAD serum-derived exosomes are taken up by N2a cells. FIG. 3A shows that cells incubated with 5×FAD serum-derived exosomes colabeled for both, GFAP and ceramide confirming that they were astrosomes. Apart from fluorescence resulting from ceramide, cells incubated with wild type exosomes did not show signals for colabeling with GFAP, suggesting that uptake of astrosomes was specific for incubation with serum exosomes from 5×FAD mice. Since there were no or only few cells that showed increased ceramide signals without being colabeled for GFAP, the data demonstrate that uptake is specific for ceramide-enriched astrosomes.

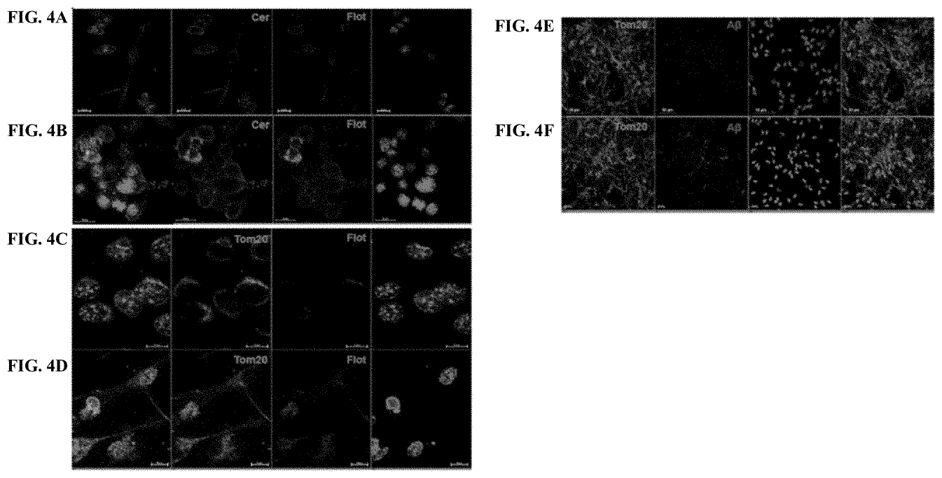

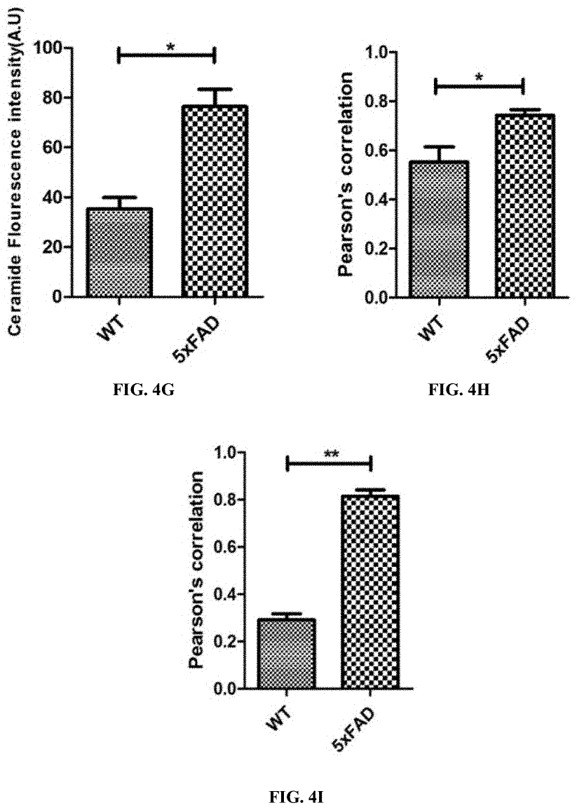

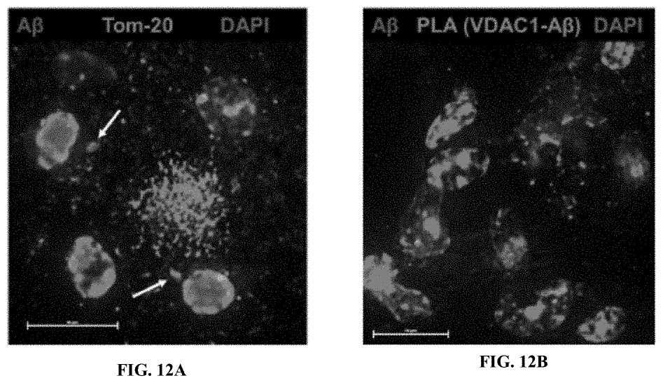

Next, it was tested whether astrosomes transported Aß into N2a cells. Using immunocytochemistry, Aß signals were detected in N2a cells incubated with 5×FAD exosomes but not with those from wild type serum (FIGS. 3D and E). The Aß signal colocalized with labeling for flotillin-2, suggesting that astrosomes delivered Aß into N2a cells. To further confirm the validity of these results, a proximity ligation assay (PLA) was used for complex formation between ceramide and Aß in cell membrane dye PKH67-labeled exosomes taken up by N2a cells (Jiang et al., 2019; Kong et al., 2018). FIGS. 11A and B shows that PLA signals colocalized with PKH67 staining and were only observed in cells incubated with 5×FAD exosomes. Uptake of serum-derived exosomes from human AD patients and healthy controls matched for sex, age, and body matrix index (BMI) was then tested. Similar to the results obtained with 5×FAD exosomes, N2a cells showed colocalization of flotillin-2 and ceramide above the background levels when incubated with exosomes from AD patients, but not with those from healthy controls (FIGS. 4A and B). This data showed that exosomes isolated from AD patient or 5×FAD serum are similar in that they transport ceramide and Aß into N2a cells.

Several studies showed that mitochondria are affected by Aß (Cha et al., 2012; Mossmann et al., 2014; Reddy & Beal, 2008). Using immunocytochemistry for Aß and Tom-20, it was shown that Aß was labeled in mitochondria of hippocampal tissue from AD patients (arrows in FIG. 12A), suggesting that Aß is transported to mitochondria in AD brain. Whether tested if Aß-associated astrosomes from 5×FAD mouse or AD patient serum were transported to mitochondria of N2a cells was tested. FIGS. 4C and D shows that flotillin-2 colocalized with Tom-20, suggesting that 5×FAD exosomes were transported to mitochondria in neural cells. Neuronally differentiated human iPS cells was used to test if neurons showed a similar uptake of exosomes as observed with N2a cells. Consistent with their function as carrier for Aß, mitochondria in human neurons incubated with AD exosomes, but not healthy control exosomes, were colabeled for Aß (FIGS. 4E and F) demonstrating that AD exosomes transport Aß to mitochondria in neurons.

### Example 4: Astrosomes Induce Aβ-VDAC1 Complex Formation, which Activates Caspases

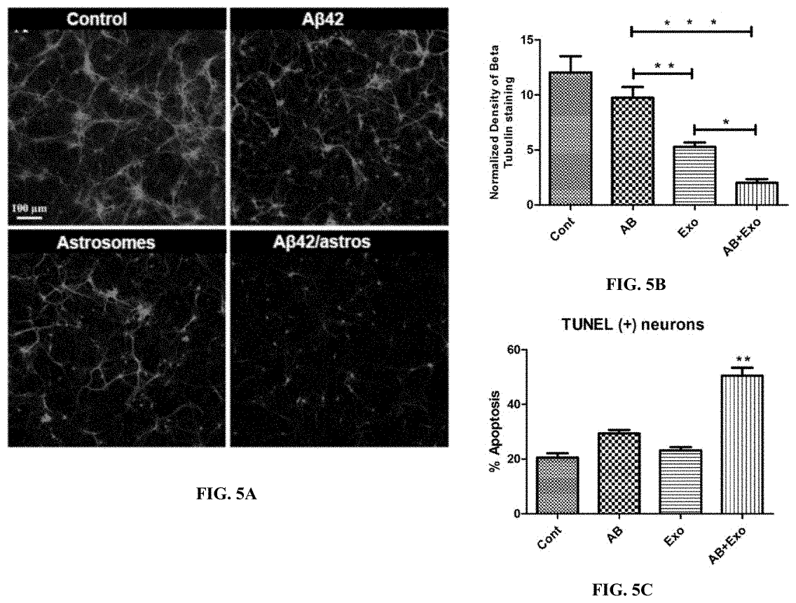

The observation that astrosomes enriched with ceramide shuttle Aß to mitochondria in neurons prompted us to investigate if Aß-associated astrosomes are neurotoxic by inducing mitochondrial damage. To test if astrosomes themselves were neurotoxic TUNEL assays were performed after incubation of primary cultured neurons from mouse brain with astrosomes, Aß, and Aß pre-incubated with astrosomes (FIG. 5). The number of TUNEL positive cells was increased by 2.6-fold when cells were incubated with Aß-associated astrosomes, concurrent with 5.9-fold enhanced fragmentation of neuronal processes as determined by ß-tubulin labeling. This result showed that astrosomes themselves were only marginally toxic, but they enhanced neurotoxicity of Aß.

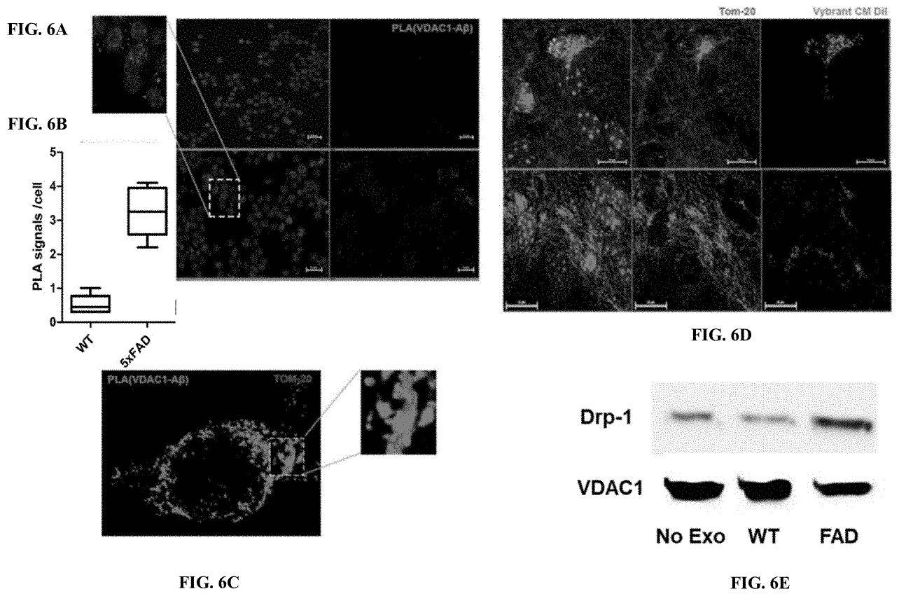

Mitochondrial dysfunction is known to be a critical factor in induction of neurotoxicity leading to neurodegeneration in AD (Cheng & Bai, 2018; Eckert, Schmitt, & Gotz, 2011; Onyango, Dennis, & Khan, 2016). One of the previously described targets for Aß is mitochondrial voltage gated anion channel 1(VDAC1), a mitochondrial gatekeeper for ADP/ATP and calcium localized in the outer mitochondrial membrane (Okada et al., 2004; Shoshan-Barmatz et al., 2010). It was tested whether astrosome-associated Aß interacted with VDAC1 and induced mitochondrial dysfunction. PLAs using antibodies to VDAC1 and Aß showed a 6-fold increase (p<0.001) in the number of signals indicating complex formation between VDAC1 and Aß when N2a cells were incubated with exosomes from 5×FAD serum as compared to those from wild type serum (FIG. 6A). FIG. 6C shows that PLA signals were colocalized with Tom-20, confirming that astrosome-associated Aß formed complexes with mitochondrial VDAC1. PLA signals for VDAC1-Aß complexes were also found in the vicinity of amyloid plaques of AD brain tissue, suggesting that VDAC1-Aß complex formation contributes to AD pathology in vivo (FIG. 12B).

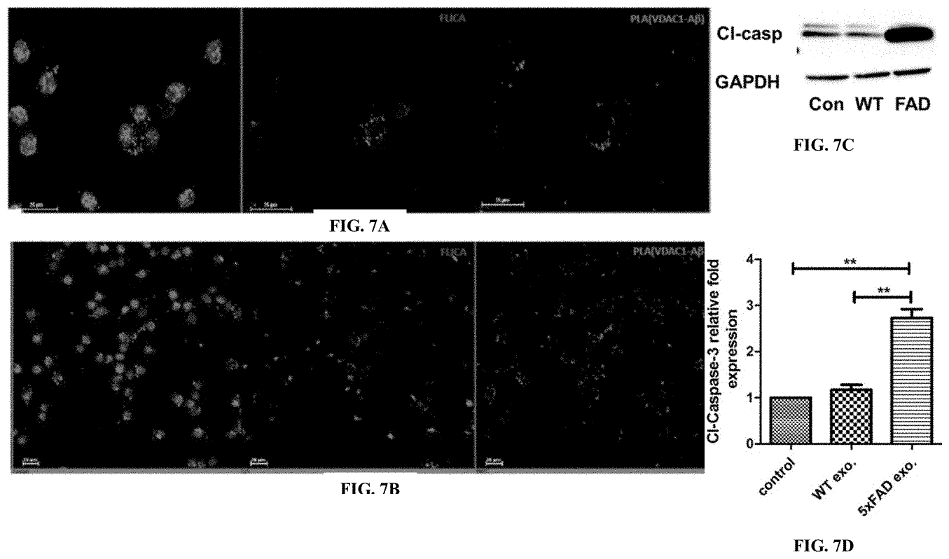

Next, it was tested if interaction of Aß with VDAC1 induced mitochondrial damage. In addition to being colocalized with PLA signals for Aß-VDAC1 complexes, labeling for Tom-20 indicated clustering of mitochondria, indicative of mitochondrial dysfunction (FIG. 6D). Consistent with this conclusion, FIG. 6E shows that serum-derived exosomes from 5×FAD mice, but not those from wild type mice, induced elevation of the mitochondrial fission protein Drp-1. It was found that PLA signals for Aß-VDAC1 complexes were colocalized with cells labeling positive for activation of caspases (FLICA assays), suggesting induction of apoptosis by exosomes from 5×FAD mice, but not wild type mice (FIG. 7A). Activation of caspases was confirmed by immunoblot analysis for cleaved caspase 3 (FIG. 7C). Similar results were obtained with serum-derived exosomes from human AD patients (FIG. 7B), indicating that Aß-associated exosomes from serum of 5×FAD and AD patients induce apoptosis. In conclusion, the results show that association of Aß with astrosomes induces mitochondrial damage and neuronal cell death.

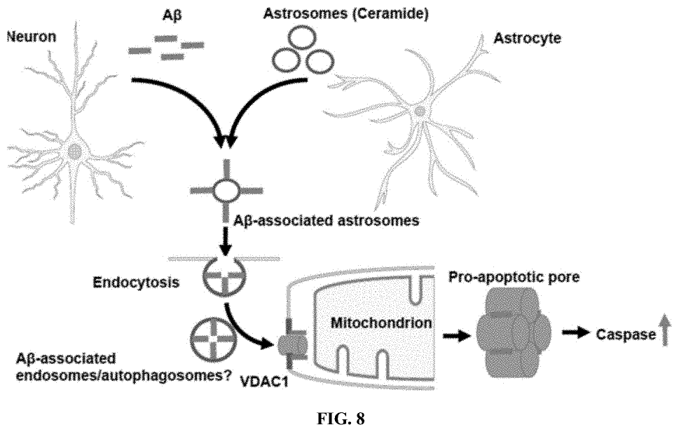

FIG. 8 shows a model for endocytotic uptake and interaction with VDAC1 at mitochondria mediated by Aβ-associated astrosomes. Aβ-associated astrosomes may either be endocytosed as vesicles or first fuse with the plasma membrane. In both cases, Aβ (FIG. 8) remains associated with ceramide (FIG. 8), probably in form of ceramide-rich platforms, a type of lipid rafts enriched with ceramide (Bieberich, 2018). The persistent association with ceramide explains why Aβ and ceramide remain colabeled after uptake of Aβ-associated exosomes into N2a cells and neurons (FIG. 11). Next, Aβ is shuttled to mitochondria, which is probably mediated by vesicular transport, either by Aβ-associated endosomes or other types of vesicular compartment such as aberrant autophagosomes (Muresan, Varvel, Lamb, & Muresan, 2009; Nixon et al., 2005; Seifert et al., 2016; Yu et al., 2005). Finally, Aβ is imported into mitochondria to interact with VDAC1, which induces a pro-apoptotic pore that leads to release of cytochrome c and activation of caspases (Smilansky et al., 2015). While interaction of Aβ with VDAC1 and formation of the pro-apoptotic pore was reported, the role of ceramide and exosomes in this process has not yet been investigated.

It will be understood that various details of the presently disclosed subject matter can be changed without departing from the scope of the subject matter disclosed herein. Furthermore, the foregoing description is for the purpose of illustration only, and not for the purpose of limitation.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

Bartolome, F., de la Cueva, M., Pascual, C., Antequera, D., Fernandez, T., Gil, C., . . . Carro, E. (2018). Amyloid beta-induced impairments on mitochondrial dynamics, hippocampal neurogenesis, and memory are restored by phosphodiesterase 7 inhibition. Alzheimers Res Ther, 10(1), 24. doi:10.1186/s13195-018-0352-4
Bieberich, E. (2011). Lipid vesicle-mediated affinity chromatography using magnetic activated cell sorting (LIMACS): a novel method to analyze protein-lipid interaction. J Vis Exp(50). doi:10.3791/2657
Bieberich, E. (2018). Sphingolipids and lipid rafts: Novel concepts and methods of analysis. Chem Phys Lipids, 216, 114-131. doi:10.1016/j.chemphyslip.2018.08.003
Bieberich, E., Hu, B., Silva, J., MacKinnon, S., Yu, R. K., Fillmore, H., . . . Ottenbrite, R. M. (2002). Synthesis and characterization of novel ceramide analogs for induction of apoptosis in human cancer cells. Cancer Lett, 181(1), 55-64. doi:10.1016/s0304-3835(02)00049-6
Bieberich, E., Kawaguchi, T., & Yu, R. K. (2000). N-acylated serinol is a novel ceramide mimic inducing apoptosis in neuroblastoma cells. J Biol Chem, 275(1), 177-181.
Brier, M. R., Gordon, B., Friedrichsen, K., McCarthy, J., Stern, A., Christensen, J., . . . Ances, B. M. (2016). Tau and Abeta imaging, CSF measures, and cognition in Alzheimer's disease. Sci Transl Med, 8(338), 338ra366. doi:10.1126/scitranslmed.aaf2362
Casson, L., Howell, L., Mathews, L. A., Ferrer, M., Southall, N., Guha, R., . . . Beverly, L. J. (2013). Inhibition of ceramide metabolism sensitizes human leukemia cells to inhibition of BCL2-like proteins. PLoS One, 8(1), e54525. doi:10.1371/journal.pone.0054525
Cha, M. Y., Han, S. H., Son, S. M., Hong, H. S., Choi, Y. J., Byun, J., & Mook-Jung, I. (2012). Mitochondria-specific accumulation of amyloid beta induces mitochondrial dysfunction leading to apoptotic cell death. PLoS One, 7(4), e34929. doi:10.1371/journal.pone.0034929
Chen, W. W., Zhang, X., & Huang, W. J. (2016). Role of neuroinflammation in neurodegenerative diseases (Review). Mol Med Rep, 13(4), 3391-3396. doi:10.3892/mmr.2016.4948
Cheng, Y., & Bai, F. (2018). The Association of Tau With Mitochondrial Dysfunction in Alzheimer's Disease. Front Neurosci, 12, 163. doi:10.3389/fnins.2018.00163
Colombo, M., Moita, C., van Niel, G., Kowal, J., Vigneron, J., Benaroch, P., . . . Raposo, G. (2013). Analysis of ESCRT functions in exosome biogenesis, composition and secretion highlights the heterogeneity of extracellular vesicles. J Cell Sci, 126(Pt 24), 5553-5565. doi:10.1242/jcs.128868
Colombo, M., Raposo, G., & Thery, C. (2014). Biogenesis, secretion, and intercellular interactions of exosomes and other extracellular vesicles. Annu Rev Cell Dev Biol, 30, 255-289. doi:10.1146/annurev-cellbio-101512-122326
Cuadrado-Tejedor, M., Vilarino, M., Cabodevilla, F., Del Rio, J., Frechilla, D., & Perez-Mediavilla, A. (2011). Enhanced expression of the voltage-dependent anion channel 1 (VDAC1) in Alzheimer's disease transgenic mice: an insight into the pathogenic effects of amyloid-beta. J Alzheimers Dis, 23(2), 195-206. doi:10.3233/JAD-2010-100966
Dinkins, M. B., Dasgupta, S., Wang, G., Zhu, G., & Bieberich, E. (2014). Exosome reduction in vivo is associated with lower amyloid plaque load in the SXFAD mouse model of Alzheimer's disease. Neurobiol Aging, 35(8), 1792-1800. doi:10.1016/j.neurobiolaging.2014.02.012
Dinkins, M. B., Dasgupta, S., Wang, G., Zhu, G., He, Q., Kong, J. N., & Bieberich, E. (2015). The SXFAD Mouse Model of Alzheimer's Disease Exhibits an Age-Dependent Increase in Anti-Ceramide IgG and Exogenous Administration of Ceramide Further Increases Anti-Ceramide Titers and Amyloid Plaque Burden. J Alzheimers Dis, 46(1), 55-61. doi:10.3233/JAD-150088
Dinkins, M. B., Enasko, J., Hernandez, C., Wang, G., Kong, J., Helwa, I., . . . Bieberich, E. (2016). Neutral Sphingomyelinase-2 Deficiency Ameliorates Alzheimer's Disease Pathology and Improves Cognition in the SXFAD Mouse. J Neurosci, 36(33), 8653-8667. doi:10.1523/JNEUROSCI.1429-16.2016
Dinkins, M. B., Wang, G., & Bieberich, E. (2017). Sphingolipid-Enriched Extracellular Vesicles and Alzheimer's Disease: A Decade of Research. J Alzheimers Dis, 60(3), 757-768. doi:10.3233/JAD-160567
Eckert, A., Schmitt, K., & Gotz, J. (2011). Mitochondrial dysfunction—the beginning of the end in Alzheimer's disease? Separate and synergistic modes of tau and amyloid-beta toxicity. Alzheimers Res Ther, 3(2), 15. doi:10.1186/alzrt74
Eitan, E., Hutchison, E. R., Marosi, K., Comotto, J., Mustapic, M., Nigam, S. M., . . . Mattson, M. P. (2016). Extracellular Vesicle-Associated Abeta Mediates Trans-Neuronal Bioenergetic and Ca(2+)-Handling Deficits in Alzheimer's Disease Models. NPJ Aging Mech Dis, 2. doi:10.1038/npjamd.2016.19
Elsherbini, A., & Bieberich, E. (2018). Ceramide and Exosomes: A Novel Target in Cancer Biology and Therapy. Adv Cancer Res, 140, 121-154. doi:10.1016/bs.acr.2018.05.004
Enderle, D., Spiel, A., Coticchia, C. M., Berghoff, E., Mueller, R., Schlumpberger, M., . . . Noerholm, M. (2015). Characterization of RNA from Exosomes and Other Extracellular Vesicles Isolated by a Novel Spin Column-Based Method. PLoS One, 10(8), e0136133. doi:10.1371/journal.pone.0136133
Fenteany, G., Standaert, R. F., Reichard, G. A., Corey, E. J., & Schreiber, S. L. (1994). A beta-lactone related to lactacystin induces neurite outgrowth in a neuroblastoma cell line and inhibits cell cycle progression in an osteosarcoma cell line. Proc Natl Acad Sci USA, 91(8), 3358-3362. doi:10.1073/pnas.91.8.3358
Fernandez-Echevarria, C., Diaz, M., Ferrer, I., Canerina-Amaro, A., & Marin, R. (2014). Abeta promotes VDAC1 channel dephosphorylation in neuronal lipid rafts. Relevance to the mechanisms of neurotoxicity in Alzheimer's disease. Neuroscience, 278, 354-366. doi:10.1016/j.neuroscience.2014.07.079
Fiandaca, M. S., Kapogiannis, D., Mapstone, M., Boxer, A., Eitan, E., Schwartz, J. B., . . . Goetzl, E. J. (2015). Identification of preclinical Alzheimer's disease by a profile of pathogenic proteins in neurally derived blood exosomes: A case-control study. Alzheimers Dement, 11(6), 600-607 e601. doi:10.1016/j.jalz.2014.06.008
Flis, V. V., & Daum, G. (2013). Lipid transport between the endoplasmic reticulum and mitochondria. Cold Spring Harb Perspect Biol, 5(6). doi:10.1101/cshperspect.a013235 Funato, K., & Riezman, H. (2001). Vesicular and nonvesicular transport of ceramide from ER to the Golgi apparatus in yeast. J Cell Biol, 155(6), 949-959. doi:10.1083/jcb.200105033
Goetzl, E. J., Boxer, A., Schwartz, J. B., Abner, E. L., Petersen, R. C., Miller, B. L., & Kapogiannis, D. (2015). Altered lysosomal proteins in neural-derived plasma exosomes in preclinical Alzheimer disease. Neurology, 85(1), 40-47. doi:10.1212/WNL.0000000000001702
Goetzl, E. J., Kapogiannis, D., Schwartz, J. B., Lobach, I. V., Goetzl, L., Abner, E. L., . . . Miller, B. L. (2016). Decreased synaptic proteins in neuronal exosomes of frontotemporal dementia and Alzheimer's disease. FASEB J, 30(12), 4141-4148. doi:10.1096/fj.0.201600816R
Hanada, K. (2010). Intracellular trafficking of ceramide by ceramide transfer protein. Proc Jpn Acad Ser B Phys Biol Sci, 86(4), 426-437. doi:10.2183/pjab.86.426
Helwa, I., Cai, J., Drewry, M. D., Zimmerman, A., Dinkins, M. B., Khaled, M. L., . . . Liu, Y. (2017). A Comparative Study of Serum Exosome Isolation Using Differential Ultracentrifugation and Three Commercial Reagents. PLoS One, 12(1), e0170628. doi:10.1371/journal.pone.0170628
Ishida, N., Ishihara, Y., Ishida, K., Tada, H., Funaki-Kato, Y., Hagiwara, M., . . . Matsushita, K. (2017). Periodontitis induced by bacterial infection exacerbates features of Alzheimer's disease in transgenic mice. NPJ Aging Mech Dis, 3, 15. doi:10.1038/s41514-017-0015-x
Jiang, X., Zhu, Z., Qin, H., Tripathi, P., Zhong, L., Elsherbini, A., . . . Bieberich, E. (2019). Visualization of Ceramide-Associated Proteins in Ceramide-Rich Platforms Using a Cross-Linkable Ceramide Analog and Proximity Ligation Assays With Anti-ceramide Antibody. Frontiers in Cell and Developmental Biology, 7(166). doi:10.3389/fcell.2019.00166
Kong, J. N., Zhu, Z., Itokazu, Y., Wang, G., Dinkins, M. B., Zhong, L., . . . Bieberich, E. (2018). Novel function of ceramide for regulation of mitochondrial ATP release in astrocytes. J Lipid Res, 59(3), 488-506. doi:10.1194/jlr.M081877
Krishtal, J., Bragina, O., Metsla, K., Palumaa, P., & Tougu, V. (2017). In situ fibrillizing amyloid-beta 1-42 induces neurite degeneration and apoptosis of differentiated SH-SY5Y cells. PLoS One, 12(10), e0186636. doi:10.1371/journal.pone.0186636
Lewis, A. C., Wallington-Beddoe, C. T., Powell, J. A., & Pitson, S. M. (2018). Targeting sphingolipid metabolism as an approach for combination therapies in haematological malignancies. Cell Death Discov, 4, 4. doi:10.1038/s41420-018-0075-0
Li, X., Zhang, X., Ladiwala, A. R., Du, D., Yadav, J. K., Tessier, P. M., . . . Buxbaum, J. N. (2013). Mechanisms of transthyretin inhibition of beta-amyloid aggregation in vitro. J Neurosci, 33(50), 19423-19433. doi:10.1523/JNEUROSCI.2561-13.2013
Lim, C. Z. J., Zhang, Y., Chen, Y., Zhao, H., Stephenson, M. C., Ho, N. R. Y., . . . Shao, H. (2019). Subtyping of circulating exosome-bound amyloid beta reflects brain plaque deposition. Nat Commun, 10(1), 1144. doi:10.1038/s41467-019-09030-2
Manczak, M., Calkins, M. J., & Reddy, P. H. (2011). Impaired mitochondrial dynamics and abnormal interaction of amyloid beta with mitochondrial protein Drp1 in neurons from patients with Alzheimer's disease: implications for neuronal damage. Hum Mol Genet, 20(13), 2495-2509. doi:10.1093/hmg/ddr139
Manczak, M., & Reddy, P. H. (2012). Abnormal interaction of VDAC1 with amyloid beta and phosphorylated tau causes mitochondrial dysfunction in Alzheimer's disease. Hum Mol Genet, 21(23), 5131-5146. doi:10.1093/hmg/dds360
Manczak, M., Sheiko, T., Craigen, W. J., & Reddy, P. H. (2013). Reduced VDAC1 protects against Alzheimer's disease, mitochondria, and synaptic deficiencies. J Alzheimers Dis, 37(4), 679-690. doi:10.3233/JAD-130761
Mossmann, D., Vogtle, F. N., Taskin, A. A., Teixeira, P. F., Ring, J., Burkhart, J. M., . . . Meisinger, C. (2014). Amyloid-beta peptide induces mitochondrial dysfunction by inhibition of preprotein maturation. Cell Metab, 20(4), 662-669. doi:10.1016/j.cmet.2014.07.024
Muresan, V., Varvel, N. H., Lamb, B. T., & Muresan, Z. (2009). The cleavage products of amyloid-beta precursor protein are sorted to distinct carrier vesicles that are independently transported within neurites. J Neurosci, 29(11), 3565-3578. doi:10.1523/JNEUROSCI.2558-08.2009
Nisbet, R. M., & Gotz, J. (2018). Amyloid-beta and Tau in Alzheimer's Disease: Novel Pathomechanisms and Non-Pharmacological Treatment Strategies. J Alzheimers Dis, 64(s1), 5517-5527. doi:10.3233/JAD-179907
Nixon, R. A., Wegiel, J., Kumar, A., Yu, W. H., Peterhoff, C., Cataldo, A., & Cuervo, A. M. (2005). Extensive involvement of autophagy in Alzheimer disease: an immuno-electron microscopy study. J Neuropathol Exp Neurol, 64(2), 113-122. doi:10.1093/jnen/64.2.113
Okada, S. F., O'Neal, W. K., Huang, P., Nicholas, R. A., Ostrowski, L. E., Craigen, W. J., . . . Boucher, R. C. (2004). Voltage-dependent anion channel-1 (VDAC-1) contributes to ATP release and cell volume regulation in murine cells. J Gen Physiol, 124(5), 513-526. doi:10.1085/jgp.200409154
Onyango, I. G., Dennis, J., & Khan, S. M. (2016). Mitochondrial Dysfunction in Alzheimer's Disease and the Rationale for Bioenergetics Based Therapies. Aging Dis, 7(2), 201-214. doi:10.14336/AD.2015.1007
Pagano, R. E., Sepanski, M. A., & Martin, O. C. (1989). Molecular trapping of a fluorescent ceramide analogue at the Golgi apparatus of fixed cells: interaction with endogenous lipids provides a trans-Golgi marker for both light and electron microscopy. J Cell Biol, 109(5), 2067-2079. doi:10.1083/jcb.109.5.2067
Perez-Gonzalez, R., Gauthier, S. A., Kumar, A., & Levy, E. (2012). The exosome secretory pathway transports amyloid precursor protein carboxyl-terminal fragments from the cell into the brain extracellular space. J Biol Chem, 287(51), 43108-43115. doi:10.1074/jbc.M112.404467
Rad, S. K., Arya, A., Karimian, H., Madhavan, P., Rizwan, F., Koshy, S., & Prabhu, G. (2018). Mechanism involved in insulin resistance via accumulation of beta-amyloid and neurofibrillary tangles: link between type 2 diabetes and Alzheimer's disease. Drug Des Devel Ther, 12, 3999-4021. doi:10.2147/DDDT.S173970
Rapoport, M., Dawson, H. N., Binder, L. I., Vitek, M. P., & Ferreira, A. (2002). Tau is essential to beta-amyloid-induced neurotoxicity. Proc Natl Acad Sci USA, 99(9), 6364-6369. doi:10.1073/pnas.092136199
Reddy, P. H. (2013). Amyloid beta-induced glycogen synthase kinase 3beta phosphorylated VDAC1 in Alzheimer's disease: implications for synaptic dysfunction and neuronal damage. Biochim Biophys Acta, 1832(12), 1913-1921. doi:10.1016/j.bbadis.2013.06.012
Reddy, P. H., & Beal, M. F. (2008). Amyloid beta, mitochondrial dysfunction and synaptic damage: implications for cognitive decline in aging and Alzheimer's disease. Trends Mol Med, 14(2), 45-53. doi:10.1016/j.molmed.2007.12.002
Sardar Sinha, M., Ansell-Schultz, A., Civitelli, L., Hildesjo, C., Larsson, M., Lannfelt, L., . . . Hallbeck, M. (2018). Alzheimer's disease pathology propagation by exosomes containing toxic amyloid-beta oligomers. Acta Neuropathol, 136(1), 41-56. doi:10.1007/s00401-018-1868-1
Sarkar, P., Zaja, I., Bienengraeber, M., Rarick, K. R., Terashvili, M., Canfield, S., . . . Harder, D. R. (2014). Epoxyeicosatrienoic acids pretreatment improves amyloid beta-induced mitochondrial dysfunction in cultured rat hippocampal astrocytes. Am J Physiol Heart Circ Physiol, 306(4), H475-484. doi:10.1152/ajpheart.00001.2013
Schmitz-Peiffer, C. (2010). Targeting ceramide synthesis to reverse insulin resistance. Diabetes, 59(10), 2351-2353. doi:10.2337/db10-0912
Seifert, B., Eckenstaler, R., Ronicke, R., Leschik, J., Lutz, B., Reymann, K., . . . Brigadski, T. (2016). Amyloid-Beta Induced Changes in Vesicular Transport of BDNF in Hippocampal Neurons. Neural Plast, 2016, 4145708. doi:10.1155/2016/4145708
Serrano-Pozo, A., Frosch, M. P., Masliah, E., & Hyman, B. T. (2011). Neuropathological alterations in Alzheimer disease. Cold Spring Harb Perspect Med, 1(1), a006189. doi:10.1101/cshperspect.a006189
Sharples, R. A., Vella, L. J., Nisbet, R. M., Naylor, R., Perez, K., Barnham, K. J., . . . Hill, A. F. (2008). Inhibition of gamma-secretase causes increased secretion of amyloid precursor protein C-terminal fragments in association with exosomes. FASEB J, 22(5), 1469-1478. doi:10.1096/fj.0.07-9357com
Shi, M., Liu, C., Cook, T. J., Bullock, K. M., Zhao, Y., Ginghina, C., . . . Zhang, J. (2014). Plasma exosomal alpha-synuclein is likely CNS-derived and increased in Parkinson's disease. Acta Neuropathol, 128(5), 639-650. doi:10.1007/s00401-014-1314-y
Shoshan-Barmatz, V., De Pinto, V., Zweckstetter, M., Raviv, Z., Keinan, N., & Arbel, N. (2010). VDAC, a multi-functional mitochondrial protein regulating cell life and death. Mol Aspects Med, 31(3), 227-285. doi:10.1016/j.mam.2010.03.002
Skotland, T., Sandvig, K., & Llorente, A. (2017). Lipids in exosomes: Current knowledge and the way forward. Prog Lipid Res, 66, 30-41. doi:10.1016/j.plipres.2017.03.001
Smilansky, A., Dangoor, L., Nakdimon, I., Ben-Hail, D., Mizrachi, D., & Shoshan-Barmatz, V. (2015). The Voltage-dependent Anion Channel 1 Mediates Amyloid beta Toxicity and Represents a Potential Target for Alzheimer Disease Therapy. J Biol Chem, 290(52), 30670-30683. doi:10.1074/jbc.M115.691493
Taverna, S., Giallombardo, M., Pucci, M., Flugy, A., Manno, M., Raccosta, S., . . . Alessandro, R. (2015). Curcumin inhibits in vitro and in vivo chronic myelogenous leukemia cells growth: a possible role for exosomal disposal of miR-21. Oncotarget, 6(26), 21918-21933. doi:10.18632/oncotarget.4204
Tu, S., Okamoto, S., Lipton, S. A., & Xu, H. (2014). Oligomeric Abeta-induced synaptic dysfunction in Alzheimer's disease. Mol Neurodegener, 9, 48. doi:10.1186/1750-1326-9-48
Wang, G., Dinkins, M., He, Q., Zhu, G., Poirier, C., Campbell, A., . . . Bieberich, E. (2012). Astrocytes secrete exosomes enriched with proapoptotic ceramide and prostate apoptosis response 4 (PAR-4): potential mechanism of apoptosis induction in Alzheimer disease (AD). J Biol Chem, 287(25), 21384-21395. doi:10.1074/jbc.M112.340513
Yamaji, T., & Hanada, K. (2015). Sphingolipid metabolism and interorganellar transport: localization of sphingolipid enzymes and lipid transfer proteins. Traffic, 16(2), 101-122. doi:10.1111/tra.12239
Yu, W. H., Cuervo, A. M., Kumar, A., Peterhoff, C. M., Schmidt, S. D., Lee, J. H., . . . Nixon, R. A. (2005). Macroautophagy—a novel Beta-amyloid peptide-generating pathway activated in Alzheimer's disease. J Cell Biol, 171(1), 87-98. doi:10.1083/jcb.200505082
Zhang, Y., Liu, Y., Liu, H., & Tang, W. H. (2019). Exosomes: biogenesis, biologic function and clinical potential. Cell Biosci, 9, 19. doi:10.1186/s13578-019-0282-2

1. A method of diagnosing and treating a subject with Alzheimer's Disease, said method comprising:
a. obtaining a blood sample from a subject;
b. purifying exosomes from the blood;
c. detecting the size of exosomes in the blood;
d. diagnosing the subject with Alzheimer's Disease when exosomes over 100 nm in size comprise at least 30% of the total population of exosomes; and
e. administering an effective amount of a ceramide analogue to the subject diagnosed with Alzheimer's Disease.

2. The method of claim 1, wherein the ceramide analogue is N-oleoyl serinol (S18).

3. The method of claim 2, wherein the effective amount is about 50 micromolar.

4. The method of claim 1, wherein the subject is a human

5. A method of diagnosing and treating a subject with Alzheimer's Disease, said method comprising:
a. obtaining a blood sample from a subject;
b. purifying exosomes from the blood;
c. determining lipid composition of the exosomes in the subject;
d. comparing the lipid composition of the exosomes in the subject to the lipid composition of the exosomes of healthy subjects;
e. diagnosing the subject with Alzheimer's Disease when ceramide species C16:0 and C18:0 C22:0, C24:0 or C24:1 are elevated in the subject relative to the healthy subjects; and
f. administering an effective amount of a ceramide analogue to the subject diagnosed with Alzheimer's Disease.

6. The method of claim 5, wherein the ceramide analogue is N-oleoyl serinol (S18).

7. The method of claim 6, wherein the effective amount is about 50 micromolar.

8. The method of claim 5, wherein the subject is a human.

9. A method of reducing the size of exosomes in a subject, comprising: administering an effective amount of a ceramide analogue to a subject in need thereof.

10. The method of claim 9, wherein the ceramide analogue is N-oleoyl serinol (S18).

11. The method of claim 9, wherein the effective amount is about 50 micromolar.

12. The method of claim 9, wherein the subject is a human.

13. The method of claim 9, wherein the size is reduced by inhibiting protein binding to the exosomes.

14. The method of claim 13, wherein the protein is Aβ.
